(19) United States
(12) Patent Application Publication
(10) Pub. No.: US 2019/0070335 A1
(43) Pub. Date: Mar. 7, 2019

(54) AMINION BASED CONDUIT TISSUES

(71) Applicant: Marianne KARAGIANIS, Los Angeles, CA (US)

(72) Inventor: Marianne KARAGIANIS, Los Angeles, CA (US)

(21) Appl. No.: 16/182,330

(22) Filed: Nov. 6, 2018

Related U.S. Application Data
(63) Continuation of application No. 15/583,906, filed on May 1, 2017, now abandoned.

(51) Int. Cl. A61L 27/36; A61L 2/00

(52) U.S. Cl. CPC A61L 27/3604; A61L 2430/32; A61L 2202/21; A61L 2/0094; A61L 27/3691; A61L 27/3687; A61L 27/3675

(57) ABSTRACT

Described herein is the method of preparation for amnion-based tissue conduits. Amnion based tissue conduits are obtained from placental and umbilical cord tissue. Wherein said tissues are separated into one or more layers of amnion, chorion and umbilical cord and incised into predetermined measurements. By incising tissues, the quantified measurements will be less readily able to degrade bioactive properties during the predetermined duration of exposure of super critical carbon dioxide sterilization and a disinfectant wash of amnion, chorion and umbilical cord.

### CROSS-REFERENCE TO RELATED APPLICATION

This application is a continuation of U.S. patent application Ser. No. 15/583,906 filed May 1, 2017, the content of which is incorporated herein by reference in its entirety.

### BACKGROUND

Twenty million Americans suffer from peripheral nerve injury caused by trauma and medical disorders. Nerve injuries result in approximately $150 billion spent in annual healthcare dollars in the United States. The majority of peripheral nerve injuries occur in the upper limb and are from traumatic causes. These injuries disproportionately afflict young healthy civilians and military officers who are most at risk of traumatic injuries. Severe nerve injury has a devastating impact on a patients' quality of life. Typical symptoms are sensory and motor function defects that can result in complete paralysis of the affected limb or development of intractable neuropathic pain

There are currently only two choices for conduit nerve repair and new developments in tissue engineering promise strong alternatives. Autologous nerve graft is the gold standard of nerve graft repair, it has several disadvantages, including the need for an extra incision, loss of donor nerve function, mismatch in size between the donor nerve and the injured nerve, and a limited availability of donor nerve. The second is to use a Xenograft conduit comprised of bovine tendon collagen to bridge a gap between severed nerves. However new research and development in the field of placental tissues and umbilical cord offer a promising alternative for a conduit derived from an allogenic extra cellular matrix. There is extensive and compelling evidence that the success of peripheral nerve regeneration depends on the extracellular matrix.

Tissue engineering techniques can be powerful modalities to improve the effectiveness of nerve conduit bridging. Support Cells have bioactivity and can produce nerve growth factors. Adherent molecules on the surface of Support Cells can secrete extracellular matrix and guide the growth of axons. Neurotrophic factors secreted by Support Cells may be the most important factors in the microenvironment for regenerating axons.

Fetal tissues represent the most primitive source of mesenchymal stem cells (MSCs). These tissues are ordinarily discarded following birth and are therefore in abundant supply. Tissue age rarely exceeds 42-wk and therefore, in comparison to other adult sources, cells have accumulated less genetic damage due to age, environment and disease. Stem cells can be harvested from amniotic membrane, amniotic fluid, umbilical cord cells, umbilical cord blood and Wharton's jelly. These cells are readily expandable in culture and possess the ability to differentiate into neural phenotype. Umbilical cord-derived mesenchymal stem cells have also been shown to improve outcomes following crush and transection injuries in rodent models. Fetal tissue represents a promising alternative source of stem cells. However the use of autologous cells following injury is currently impractical. The use of allogeneic cells and their associated immunoreactivity are obstacles that are not encountered with other adult sources such as adipose tissue. Widespread banking of fetal products following birth offers a solution to this problem.

### Background of Super Critical Carbon Dioxide for Tissue Processing

The preparation, preservation, and storage conditions of amniotic membranes (AMs) are extremely important. Numerous techniques and methodologies have been reported, including treatment with chemical detergents, gamma irradiation, and preservation in glycerol. Cryopreservation of tissue in glycerol is a commonly used technique (e.g., BioTissue, Inc., Miami, Fla.) because the ECM proteins and growth factors inherent to placental and umbilical cord tissues are maintained. However, cryopreservation with glycerol does not generate a sterilized product. Exposure to gamma radiation is an effective method of sterilization, but results in severe degradation of the collagenous stroma of the AM. Therefore, careful selection of the processing and sterilization procedures for placental and umbilical cord tissues are of paramount importance for the preservation of key molecules unique to the tissue matrix. An alternative to these traditional tissue processing techniques involves the use of supercritical carbon dioxide (SCCO2). The term supercritical fluid denotes a substance that, at temperature and pressure conditions above its critical point, simultaneously exhibits gaseous viscosity and diffusion properties, but liquid densities and dissolution properties. Such characteristics can easily be manipulated with small changes in temperature and pressure, thus making supercritical fluids applicable to many industrial and laboratory processes.

Of particular interest is the use of SCCO2 in biomedical applications for processing both hard and soft human tissues for transplantation. Specifically, SCCO2 has been used for the successful dilapidation of bone. Researchers found that upon exposure to SCCO2 (with the addition of hydrogen peroxide), bone cells as well as the lipid fraction of the bone could be successfully removed. Others have used SCCO2 to remove cell nuclei from soft tissue as well. In a study of porcine aortas exposed to SCCO2, researchers were able to remove the nuclear material with 20 min of exposure.

SCCO2 can also be used to terminally sterilize medical devices, implants, and allograft tissues. After 1 hour of exposure to SCCO2, spore preparations of Bacillus subtilis and Bacillus stearothermophilus were inactivated and a sterility assurance level (SAL) of 10-6 (reduction in bacterial spore colony forming units [CFUs]) was achieved. Scanning electron microscopy (SEM) revealed that the bacteria exposed to SCCO2 remained intact; however, the lipid bilayer and internal cell structures were indistinguishable. Such results suggest that the mechanism of bacterial inactivation occurs through intracellular acidification due to enhanced mass transfer of CO2 and disruption of the phospholipid bilayer.17

The use of SCCO2 for preparing tissues for transplantation and other clinical applications is only beginning to be realized. SCCO2 makes an attractive solvent for tissue processing for many reasons. First, SCCO2 has low viscosity and high diffusion coefficients that allow it to penetrate solid microporous matrices, such as tissue ECM. In addition, CO2 has relatively low critical coordinates (i.e., 93.2-98.7 atm and 35-39 C.), making it ideal for delicate biological tissues. The low temperature of the process and the stability of CO2 allow unwanted compounds, such as blood and lipids, to be extracted without compromising the physiological properties and mechanical integrity of the tissue.18 Furthermore, SCCO2 is relatively nontoxic, thus allowing for maintained biocompatibility upon transplantation. The preservation of these features make placental and umbilical cord tissue appealing for use in numerous clinical and tissue engineering applications.

### SUMMARY

The foregoing, and other features and advantages of the invention, will be apparent from the following, more particular description of the method of preparation thereof of the invention, the accompanying drawings, and the claims.

A method of preparation of an amnion-based conduit tissue comprising the steps of, first, obtaining tissue from placental tissue and umbilical cord tissue and separating placental and umbilical cord tissue into one or more layers of amnion, one or more layers of chorion, and one or more layers of umbilical cord tissue. Next, the one or more layers of amnion, the one or more layers of chorion, and the one or more layers of umbilical cord tissue are incised into measurements for sterilization and cleaning. This step is followed by sterilizing in the presence of supercritical carbon dioxide fluid in a predetermined amount of time without altering integrity of native bioactive properties and cleaning with a disinfectant without altering integrity of native bioactive properties. Amnion based tissues are verified to contain bioactive properties native to amnion followed by laminating the one or more layers of amnion, the one or more layers of chorion, and the one or more layers of umbilical cord tissue to obtain an air tight seal and circularizing to obtain a hollow conduit.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a perspective view of the amnion based conduit, according to an embodiment of the present invention.

### DETAILED DESCRIPTION

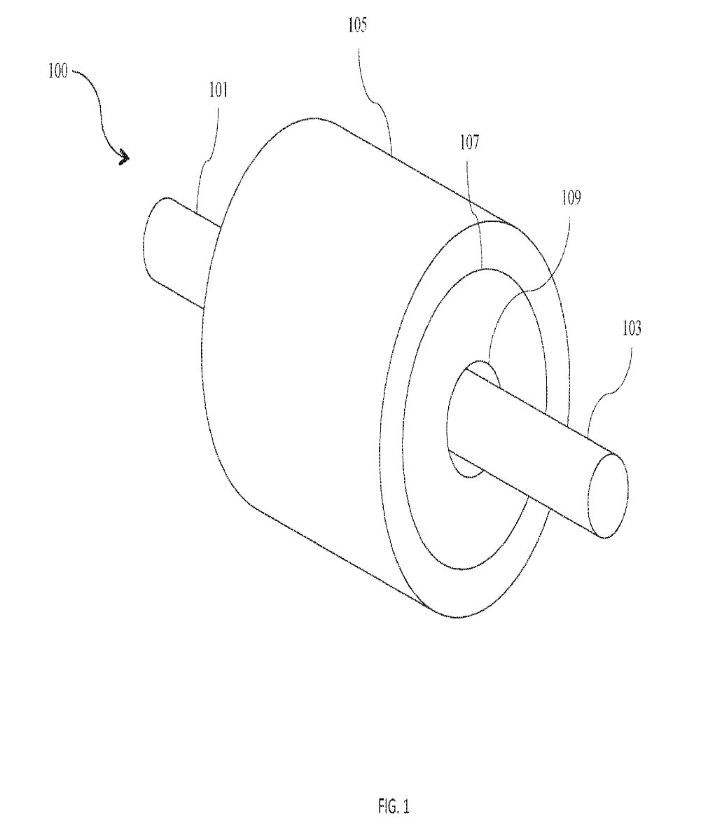

The method of preparation of the present invention may be understood by referring to FIG. 1, wherein like reference numerals refer to like elements.

Before the present articles and methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting. In this specification and in the claims that follow, reference will be made to a number of terms that shall be defined to have the following meanings.

Thus, for example, reference to “bioactive properties” includes mixtures of two or more such properties, and the like, “verify” or “verifying” or “verification” means that the subsequently described amnion based conduit tissues are taken into analysis, and that the method of analysis includes those analysis specific to what is described herein, and how the methods of analysis occurs in order to make a determination, “altering the integrity” means that the bioactive properties native to placental and umbilical cord tissues based upon one or more methods of verification to show a minimizing effect or degradation of collagens I, III, IV, V, VI, interleukin cytokines (IL-6, IL-10), growth factors TGF-B, VEGF, PDGF, bFGF and EGF deemed inadmissible by the medical examiner.

Overview of the Process

Described herein are the method of preparation for an amnion-based conduit tissues also defined as extra cellular matrix's derived from tissues of the placenta and umbilical cord. The method of preparation is intended to preserve the native bioactive properties of amniotic membrane, chorion, and umbilical cord. The amnion-based conduit tissues are composed of at least one inner layer of amnion tissue and at least two laminated outer layers of chorion or umbilical cord tissue so as to provide structural integrity in order to bridge a gap between severed nerves. Clinical studies have shown Amniotic membrane as a known source of collagens I, III, IV, V, VI and how properties native to amniotic membrane such as interleukin Cytokine (IL-6, IL-10) have been seen to regulate inflammation, transforming growth factor beta (TGF-B) have been seen to suppress scar formation, and vascular endothelial growth factor (VEGF) have been seen to act as an agent to angiogenesis. Additionally, clinical studies further show how amniotic membrane's Platelet Derived Growth Factor (PDGF), Fibroblast Growth Factor (bFGF), and Epidermal Growth Factor (EGF) have tissue engineering properties that have been seen to constructively remodel functional tissue. Additionally, umbilical cord tissue serves as a natural conduit providing structure to the amnion-based conduit in its native state and function.

Initial Tissue Collection

The collection of donated placenta tissue originates in a hospital, where it is recovered during a healthy live Cesarean section birth. The donor, electively submits to a comprehensive donor screening process designed to provide the safest tissue possible for transplantation. The screening process tests for antibodies to the human immunodeficiency virus type 1 and type 2 (anti-HIV-1 and anti-HIV-2), hepatitis B surface antigens (HBsAg), antibodies to the hepatitis C virus (anti-HCV), antibodies to the human T-lymphotropic virus type I and type II (anti-HTLV-I and anti-HTLV-II). CMV, and syphilis, using conventional serological tests. The above list of tests is exemplary only, as more, fewer, or different tests may be desired or necessary over time or based upon the intended use of the conduits.

Based upon a review of the donor's information and screening test results, the donor will either he deemed acceptable or not. In addition, at the time of delivery, cultures are taken to determine the presence of bacteria, for example, Clostridium or Streptococcus. If the donor's information, screening tests, and the delivery cultures are all satisfactory (i.e., do not indicate any risks or indicate acceptable level of risk), the donor is approved by a medical director and the tissue specimen is designated as initially eligible for further processing and evaluation.

Human placentas and umbilical cord tissue were collected prior to the completion or obtaining of results from the screening tests and delivery cultures, such tissue is labeled and kept in quarantine. The tissue is approved for further processing only after the required screening assessments and delivery cultures, which declare the tissue safe for handling and use, are satisfied and obtains final approval from a medical director.

Material Check-in and Evaluation

Upon arrival at the processing center or laboratory, the shipment is opened and verified that the sterile shipment bag/container is still sealed and in the coolant, that the appropriate donor paperwork is present, and that the donor number on the paperwork matches the number on the sterile shipment hag containing the tissue. The sterile shipment bag containing the tissue is then stored in a refrigerator until ready for further processing.

Gross Tissue Processing:

Once tissue passes donor screening and is accepted, the amnion, chorion and umbilical cord are than isolated into independent layers carefully using dissection instrumentation under sterile conditions. Once removed, the Amnion, chorion and umbilical cord tissues were thoroughly rinsed in saline to remove remaining blood clots, and general debris. Amnion, chorion and umbilical cord were then mounted on nitrocellulose paper, which were then sealed in packages and kept frozen at −80° C. before being exposed to Super Critical Carbon Dioxide Fluid.

When the tissue is ready to be processed further, the sterile supplies necessary for processing the placental and umbilical cord tissue further are assembled and prepared in a controlled environment using sterile technique.

Processing equipment is decontaminated through sterilization according to conventional and industrial grade sterilization procedures and then introduced into the controlled environment with the use of sterile technique.

Next, the placenta and umbilical cord tissues are removed from the packages and transferred aseptically to a sterile processing basin within the controlled environment.

The sterile basin contains sterile saline solution that is at room or near room temperature. After having warmed up to the ambient temperature (approximately 10-30 minutes), the amnion, chorion and umbilical cord layers are then removed from the sterile processing basin and spread out on a processing tray ready for inspection.

The placental and umbilical cord tissue is examined for discoloration, debris or other contamination, odor, and signs of damage. The size of the tissue is also noted. A determination is made, at this point, as to whether the tissue is acceptable for further processing.

Next, if the placental and umbilical cord tissue is deemed acceptable for further processing, the placental umbilical cord tissue layers are then carefully separated with sterile dissection instruments and laid out on a sterile processing tray.

Method of Decontamination with Use of SCCO2 Exposure

Sterilization consisted of exposing the separated layers of amnion, chorion and umbilical cord tissue to Super Critical Carbon Dioxide (SCCO2) Fluid, wherein the pressure is to be held constant at 9900 kPa and temperature held constant at 35° C. Exposure of separated amnion, chorion and umbilical cord is for approximately 10 to 20 minutes. SCCO2 decontamination is a source of tissue sterilization in order to enhance the preservation of bioactive properties native to placental and umbilical cord tissues in addition to being in accordance to industrial strength sterilization procedures. Following SCCO2 sterilization, amnion, chorion and umbilical cord are rinsed with a disinfectant, such as 2 cc of peracetic acid (PAA).

Methods of Verification of Amnion Based Conduit Tissues

After sterilization, the layers of amnion, chorion and umbilical cord are then verified under one or more methods of analysis to ensure bioactive properties native to the amnion, chorion and umbilical cord have not been altered wherein Collagens I, III, IV, V, VI. Interleukin Cytokines (IL-6, IL-10), growth factors TGF-β, VEGF, PDGF, bFGF and EGF are no longer present. The methods of analysis are described herein;

1. Microscopic Analysis

Briefly, tissues were fixed in 2.5% phosphate buffered glutaraldehyde at 4° C. for 1 h, dehydrated in a series of graded alcohols (50%, 70%, 80%, 90%, 95%, and 100%) and dried using hexamethyldisilazane. The samples were then sputter coated with a thin layer (10 nm) of gold and palladium and examined on a scanning electron microscope. The structure and ultrastructure of the tissues are evaluated with fluorescent microscopy.

2. Spectroscopic Analysis,

Changes in the chemical structures of amnion-based conduit tissues after treatment with SCCO2 were evaluated using the Fourier Transform Infrared (FTIR) spectroscopy. Spectra for native and SCCO2-treated tissues were acquired. Spectral scanning in the range of 4500-400 cm-1 with a resolution of 4 cm-1 was performed and the absorbance at each wavelength recorded for all samples using software.

3. Thermodynamic Analysis

The thermal transitions of the SCCO2-treated tissues were analyzed by differential scanning calorimetry (DSC). Native and SCCO2-treated tissues were lyophilized overnight, weighed (5 mg dry weight per sample), then sealed in aluminum pans before being heated at a rate of 30° C./min over a temperature range of 25-300° C. An empty aluminum pan served as the reference for all samples tested. DSC thermograms were collected, and the temperatures at which the thermal transition peaks occurred were identified. The transition temperatures, an indicator of the resistance of a material to heat denaturation, were defined as the peak maximum of the resultant endothermic peaks. A second thermal run on the native tissue was performed to compare the transitions of a denatured tissue sample.

4. Biochemical Analyses

The amounts of hydroxyproline, type IV collagen, elastin, and glycosaminoglycans (GAGs) present in native (n=6) and SCCO2-treated tissues (n=6) were quantified with commercially available assays: hydroxyproline; type IV collagen enzyme-linked immunosorbent assay (ELISA) and Blyscan sulfated GAG assay (Biocolor), respectively. All assays were conducted following the manufacturer's recommended procedures. Briefly, tissue samples were lyophilized overnight, and the dry weight measured. For the hydroxyproline assay, the tissue was completely solubilized in 12 N HCl at 100° C. for 3 h. For the extraction of type IV collagen and the GAGs, the tissue was digested in a papain extraction reagent consisting of 0.2 M sodium phosphate buffer, sodium acetate, ethylenediaminetetraacetic acid (EDTA), cysteine HCl, and papain at 65° C. overnight. Elastin was extracted by incubating tissue samples in 0.25 M oxalic acid at 60° C. for 1 h. The elastin extraction process was repeated with fresh oxalic acid, and the two extractions were pooled for analysis. The concentrations of hydroxyproline, type IV collagen, elastin, and GAGs contained in each sample tested were determined using a standard curve of light absorbance (560, 450, 513, and 656 nm for hydroxyproline, type IV collagen, elastin, and GAGs, respectively) versus known concentrations of each protein run in parallel with the experimental samples.

The degree of collagen denaturation after SCCO2 treatment was also assessed using an a-chymotrypsin assay following previously published procedures. 22 Briefly, lyophilized tissue were incubated in 0.1 M tris HCl containing 1 mg/mL a-chymotrypsin (Sigma Aldrich), 1 mM iodoacetamide, and 1 mM ethylenediaminetetraacetic acid (Sigma Aldrich) overnight at 37° C. to digest denatured collagen within the matrix. The supernatant, containing the degraded collagen, was solubilized and the amount of hydroxyproline determined.

Amnion Based Conduit Tissue and Preparation:

The steps to harvest and prepare placental and umbilical cord material for later use as an amnion-based conduit tissue are disclosed. More detailed descriptions and discussion regarding each individual step will follow. Initially, the placenta tissue is collected from a consenting patient following an elective Cesarean surgery. The tissues are preserved and transported in conventional tissue preservation manner to a suitable processing location or facility for check-in and evaluation. Gross processing, handling, and separation of the amnion-based conduit tissue layers then takes place. After separation of placental tissue and umbilical cord tissues into one or more layers of amnion, chorion and umbilical cord take place, and cut into layers of 2.54 cm2 measurements, tissue is then decontaminated by exposure of Sterilized Critical Carbon Dioxide Fluid washed with a disinfectant, such as peracetic acid, cut and packaged, and released to the market for use by surgeons and other medical professionals in appropriate surgical procedures and for nerve repair.

In general, Amnion based conduit tissues are multilayered circular systems composed of one or more layers of amnion, chorion and umbilical cord tissues. First, placenta and umbilical cord tissues are obtained through elective donation following a healthy cesarean section. Placenta tissue comprises an amniotic membrane layer and a chorion tissue layer. Each tissue is separated into one or more layers of amnion, chorion and umbilical cord tissues. Each layer is then incised into predetermined measurements for sterilization and cleaning in the presence of super critical carbon dioxide fluid without altering the integrity of their native bioactive properties. Each tissue is cleaned with peracetic acid additive solution without altering the integrity of the native bioactive properties. The amnion-based conduit tissue is then verified to contain the native bioactive properties. Processes used to verify the tissue include microscopic, spectroscopic, thermodynamic, or biochemical chemical analyses as known in the arts. Each of the one or more layers are laminated in order to seal the multiple layers of amnion, chorion, and umbilical cord. tissue. Finally, tissues are circularized to obtain a hollow conduit to bridge the gap between a severed nerve.

Tissue Sealing of Layers and Conduit Circularization

After verification, the accepted layers determined by results of one or more methods of analysis, the amnion, chorion and umbilical cord are ready to produce an amnion-based conduit tissue. In one aspect, the amnion layer is laid on a suitable drying fixture. Multilaminated tissue grafts comprised of at least 2 or more layers of amnion, chorion or umbilical cord are produced by the methods described herein. With the use of sealing techniques such as a fibrin sealant, photo tissue bonding or laser welding, at least two or more layers of amnion, chorion and umbilical cord tissues are laminated together then circularized in order to form a hollow conduit.

The method described herein is utilized to prepare a conduit 100 bridging a severed proximal 101 and distal 103 nerve endings. An outer layer is comprised of one or more layers of umbilical cord tissue(s) 105, which surrounds a middle section of one or more layers of chorion tissue(s) 107. An inner section of one or more layers of amnion tissue(s) 109 is positioned most near the severed nerve.

The actual number of layers will depend upon the surgical need and nerve or vessel repair procedure with which the tissue graft is designed to be used for.

For example, in peripheral nerve repair procedures, a single nerve has a thickness between 0.05 to 0.25 mm, whereas a single layer of amnion has a measured thickness of 20-50 urn. Therefore, a similar thickness to the autologous nerve would be most similar to multilayer amnion conduits which have a thickness up to 2 mm and are thicker and stronger, designed to bridge a gap between severed nerves unlike that of a single layer of base amnion.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

What is claimed is:

1. A method for preparation of an amnion based conduit used to bridge a gap between severed peripheral nerves, comprising:
a. separating placental and umbilical cord tissue into one or more layers of amnion, one or more layers of chorion, and one or more layers of umbilical cord tissue;
b. incising the one or more layers of amnion, the one or more layers of chorion, and the one or more layers of umbilical cord tissue into predetermined measurements for sterilization and cleaning;
c. warming the one or more layers of amnion, the one or more layers of chorion, and the one or more layers of umbilical cord tissue to ambient room temperature for between approximately 10 minutes and 30 minutes prior to sterilizing;
d. sterilizing in the presence of industrial grade supercritical carbon dioxide fluid in a predetermined amount of time, pressure, and temperature without altering the integrity of native bioactive properties;
e. washing with a disinfectant without altering the integrity of native bioactive properties, wherein the disinfectant is an organic peracetic acid additive (PAA);
f. verifying tissue viability comprising one or more of microscopic analysis, spectroscopic analysis, thermodynamic analysis, and biochemical analysis, determined by the amounts of hydroxyproline, type IV collagen, elastin, and glycosaminoglycans present in the one or more layers of amnion tissue, one or more layers of chorion tissue, and one or more layers of umbilical cord tissue thereby confirming the native properties of amnion, chorion, and umbilical cord tissues have not been altered;
g. laminating the one or more layers of amnion, the one or more layers of chorion, and the one or more layers of umbilical cord tissue with the use of fibrin sealant or a laser welding technique; and
h. circularizing the one or more laminated layers to obtain a hollow conduit to bridge a gap between severed peripheral nerves, wherein the hollow conduit has an inner diameter of between about 1.0 mm and 2.0 cm, and wherein the hollow conduit has a wall thickness of between about 0.05 mm and 0.25 mm, thereby having a similar wall thickness to an autologous peripheral nerve;
i. wherein the amnion-based conduit is made into a scaffold which comprises inner layers circumferentially surrounded by middle layers and outer layers circumferentially surrounding the middle layers, wherein the inner layers comprise laminated layers of amnion tissue, wherein the middle layers comprise laminated layers of chorion tissue, and wherein the outer layers comprise laminated layers of umbilical cord tissue.
